(19) United States
(12) Patent Application Publication
(10) Pub. No.: US 2026/0108725 A1
(43) Pub. Date: Apr. 23, 2026

(54) COCHLEAR IMPLANTS HAVING MRI-COMPATIBLE MAGNET ASSEMBLIES AND ASSOCIATED SYSTEMS AND METHODS

(71) Applicant: ADVANCED BIONICS, LLC, Valencia, CA (US)

(72) Inventors: James George Elcoate Smith, Santa Clarita, CA (US); Sung Jin Lee, Valencia, CA (US); Daniel Alexander Alanis, Newhall, CA (US); Robert Tan, Valencia, CA (US)

(21) Appl. No.: 19/117,039

(22) PCT Filed: Oct. 11, 2022
(86) PCT No.: PCT/US2022/046330

§ 371 Date: Mar. 29, 2025

(51) Int. Cl. A61N 1/05; A61N 1/372

(52) U.S. Cl. CPC A61N 1/0541; A61N 1/37229

(57) ABSTRACT

A magnet assembly including a case defining a central axis, a magnet frame within the case and rotatable about the central axis of the case, first and second elongate magnets, located within in the frame, that are diametrically magnetized, that each define a longitudinal axis and a N-S direction, that are rotatable about the longitudinal axis relative to the frame, that are separated from one another by a fixed non-zero distance that is perpendicular to at least one of the longitudinal axes, and that are not mechanically biased to respective N-S rotational orientations, and a third elongate magnet, located between the first and second elongate magnets, that defines a longitudinal axis and a N-S direction that is perpendicular to the longitudinal axis, and that is mechanically biased to a predetermined N-S rotational orientation.

### BACKGROUND

### 1. Field

The present disclosure relates generally to implantable cochlear stimulation (or “ICS”) systems.

### 2. Description of the Related Art

ICS systems are used to help the profoundly deaf perceive a sensation of sound by directly exciting the intact auditory nerve with controlled impulses of electrical current. Ambient sound pressure waves are picked up by an externally worn microphone and converted to electrical signals. The electrical signals, in turn, are processed by a sound processor, converted to a pulse sequence having varying pulse widths, rates and/or amplitudes, and transmitted to an implanted receiver circuit of the ICS system. The implanted receiver circuit is connected to an implantable electrode array that has been inserted into the cochlea of the inner ear, and electrical stimulation current is applied to varying electrode combinations to create a perception of sound. The electrode array may, alternatively, be directly inserted into the cochlear nerve without residing in the cochlea. A representative ICS system is disclosed in U.S. Pat. No. 5,824,022, which is entitled “Cochlear Stimulation System Employing Behind-The-Ear Sound processor With Remote Control” and incorporated herein by reference in its entirety. Examples of commercially available ICS sound processors include, but are not limited to, the Harmony™ BTE sound processor, the Naida™ CI Q Series sound processor and the Neptune™ body worn sound processor, which are available from Advanced Bionics.

As alluded to above, some ICS systems include an implantable cochlear stimulator (or “cochlear implant”), a sound processor unit (e.g., a body worn processor or behind-the-ear processor), and a microphone that is part of, or is in communication with, the sound processor unit. The cochlear implant communicates with the sound processor unit and, some ICS systems include a headpiece that is in communication with both the sound processor unit and the cochlear implant. The headpiece communicates with the cochlear implant by way of a transmitter (e.g., an antenna) on the headpiece and a receiver (e.g., an antenna) on the implant. Optimum communication is achieved when the transmitter and the receiver are aligned with one another. To that end, the headpiece and the cochlear implant may include respective positioning magnets that are attracted to one another, and that maintain the position of the headpiece transmitter over the implant receiver. The implant magnet may, for example, be located within a pocket in the cochlear implant housing. The skin and subcutaneous tissue that separates the headpiece magnet and implant magnet is sometimes referred to as the “skin flap,” which is frequently 3 mm to 11 mm thick, and is sometimes thicker.

The present inventors have determined that conventional cochlear implants and stimulation systems are susceptible to improvement. For example, the magnet in some conventional cochlear implant is a disk-shaped axially magnetized magnet that has north and south magnetic dipoles which are aligned in the axial direction of the disk. Such magnets are not compatible with magnetic resonance imaging (“MRI”) systems, and may have to be surgically removed from the cochlear implant prior to the MRI procedure and then surgically replaced thereafter. Other cochlear implants include a diametrically magnetized disk-shaped magnet that is rotatable relative to the remainder of the implant about its central axis, and that has a N-S orientation which is perpendicular to the central axis. The present inventors have determined that diametrically magnetized disk-shaped magnets are less than optimal because a dominant magnetic field, such as the MRI magnetic field, that is misaligned by at least 30° or more from the N-S direction of the magnet may demagnetize the magnet or generate an amount of torque on the magnet that is sufficient to dislodge or reverse the magnet and/or dislocate the associated cochlear implant and/or cause excessive discomfort to the patient.

More recently, cochlear implants with MRI-compatible magnet apparatus (or “magnet assemblies”) have been introduced. The MRI-compatible magnet apparatus have a case defining a central axis, a frame within the case that is rotatable relative to the case about the central axis, and two or more elongate diametrically magnetized magnets that are located in the frame in close proximity to one another and that are rotatable about their respective longitudinal axis relative to the frame. This combination allows the magnets to align with three-dimensional (3D) MRI magnetic fields, regardless of field direction, which results in very low amounts of torque on the magnets. Examples of such MRI-compatible magnet apparatus may be found in U.S. Pat. Nos. 9,919,154, 10,463,849, and 10,532,209. Another proposed magnet apparatus, which includes a single elongate magnet, is described in PCT Pat. Pub. No. 2020/092185 A1.

Although such MRI-compatible magnet apparatus have proven to be a significant advance in the art, the present inventors have determined that they are susceptible to improvement. For example, relatively thick skin flaps (e.g., skin flaps of at least 12 mm) create a relatively large distance between the headpiece magnet and the rotatable magnets of MRI-compatible magnet apparatus. In the exemplary context of an MRI-compatible magnet apparatus that has only two (i.e., two and no more than two) rotatable elongate diametrically magnetized magnets and is used in conjunction with an axially magnetized headpiece magnet, the present inventors have determined that, due to the relatively weak attraction force between the headpiece magnet and implant magnets that is associated with the relatively large distance, the magnetic attraction force between the implant magnets may prevent the implant magnets from rotating into optimal alignment with the magnetic field of the headpiece magnet. In addition to reducing the headpiece retention force provided by the magnets, non-optimal alignment of the magnetic fields can result in misalignment of the headpiece and implant magnets and, accordingly, misalignment of the headpiece and implant antennas.

### SUMMARY

A magnet assembly in accordance with at least one of the present inventions may include a case defining a central axis, a magnet frame within the case and rotatable about the central axis of the case, first and second elongate magnets, located within in the frame, that are diametrically magnetized, that each define a longitudinal axis and a N-S direction, that are rotatable about the longitudinal axis relative to the frame, that are separated from one another by a fixed non-zero distance that is perpendicular to at least one of the longitudinal axes, and that are not mechanically biased to respective N-S rotational orientations, and a third elongate magnet, located between the first and second elongate magnets, that defines a longitudinal axis and a N-S direction that is perpendicular to the longitudinal axis, and that is mechanically biased to a predetermined N-S rotational orientation. A method in accordance with at least one of the present inventions may include removing a magnet or a magnet assembly from an implanted cochlear implant and installing such a magnet assembly in place of the removed magnet or magnet assembly. A system in accordance with at least one of the present inventions may include a head wearable external component, including an axially magnetized external magnet, and a cochlear implant having a cochlear lead, an implant antenna, an implant processor and such an implant magnet assembly.

There are a number of advantages associated with such methods and apparatus. Byway of example, but not limitation, the magnetic force associated with the third elongate magnet offsets some of the magnetic attraction force between the first and second elongate magnets, thereby facilitating the rotation of the first and second elongate magnets into optimal alignment with the magnetic field of an axially magnetized headpiece magnet. This results in superior retention force as well as better alignment of the headpiece and implant magnets and, accordingly, better alignment of the headpiece and implant antennas.

It should also be noted here that the use of an axially magnetized headpiece magnet, which is facilitated by the reduction in magnetic attraction between the two non-mechanically biased rotatable magnets associated with the present magnet assembly, is more magnetically efficient that a diametrically magnetized headpiece magnet due to the orientation of the magnetic field of the axially magnetized headpiece magnet. As a result, less magnetic material may be employed within a magnet assembly, there is less friction between rotating magnets and the inner surface of the case and a corresponding reduction in the torque associated with placement of the magnet apparatus into a MRI magnetic field as well as less of an MRI artifact.

The above described and many other features of the present inventions will become apparent as the inventions become better understood by reference to the following detailed description when considered in conjunction with the accompanying drawings.

### BRIEF DESCRIPTION OF THE DRAWINGS

Detailed descriptions of the exemplary embodiments will be made with reference to the accompanying drawings.

FIG. 1 is a perspective view of an implant magnet apparatus (or “magnet assembly”) in accordance with one embodiment of a present invention.

FIG. 2 is a perspective view of a portion of the implant magnet apparatus illustrated in FIG. 1.

FIG. 3 is an exploded perspective view of the implant magnet apparatus illustrated in FIG. 1.

FIG. 4 is a plan view of a portion of the implant magnet apparatus illustrated in FIG. 1.

FIG. 5 is an end view of a portion of the implant magnet apparatus illustrated in FIG. 1.

FIG. 6 is an exploded perspective view of a portion of the implant magnet apparatus illustrated in FIG. 1.

FIG. 7 is a section view taken along line 7-7 in FIG. 1.

FIG. 7A is an enlarged view of a portion of FIG. 7.

FIG. 7B is a section view in accordance with one embodiment of a present invention.

FIG. 8 is an enlarged view of a portion of the a section view illustrated in FIG. 7.

FIG. 9 is a partial section view of a system including a headpiece and an implant with the magnet apparatus illustrated in FIG. 1.

FIG. 9A is a plan view of a portion of the implant magnet apparatus illustrated in FIG. 1.

FIG. 9B is a plan view of a portion of an implant magnet apparatus in accordance with one embodiment of a present invention.

FIG. 10 is a section view similar to FIG. 9 with the implant in an MRI magnetic field.

FIG. 11 is a perspective view of an implant magnet apparatus (or “magnet assembly”) in accordance with one embodiment of a present invention.

FIG. 12 is a perspective view of a portion of the implant magnet apparatus illustrated in FIG. 11.

FIG. 13 is a plan view of a portion of the implant magnet apparatus illustrated in FIG. 11.

FIG. 14 is an exploded perspective view of the implant magnet apparatus illustrated in FIG. 11.

FIG. 15 is an exploded perspective view of the implant magnet apparatus illustrated in FIG. 11.

FIG. 16 is a section view taken along line 16-16 in FIG. 11.

FIG. 17 is a perspective view of an implant magnet apparatus (or “magnet assembly”) in accordance with one embodiment of a present invention.

FIG. 18 is a plan view of a portion of the implant magnet apparatus illustrated in FIG. 17.

FIG. 19 is an end view of a portion of the implant magnet apparatus illustrated in FIG. 17.

FIG. 20 is a top view of a cochlear implant in accordance with one embodiment of a present invention.

FIG. 21 is a block diagram of a cochlear implant system in accordance with one embodiment of a present invention.

FIG. 22 is a flow chart showing a method in accordance with one embodiment of a present invention.

### DETAILED DESCRIPTION OF THE EXEMPLARY EMBODIMENTS

The following is a detailed description of the best presently known modes of carrying out the inventions. This description is not to be taken in a limiting sense, but is made merely for the purpose of illustrating the general principles of the inventions.

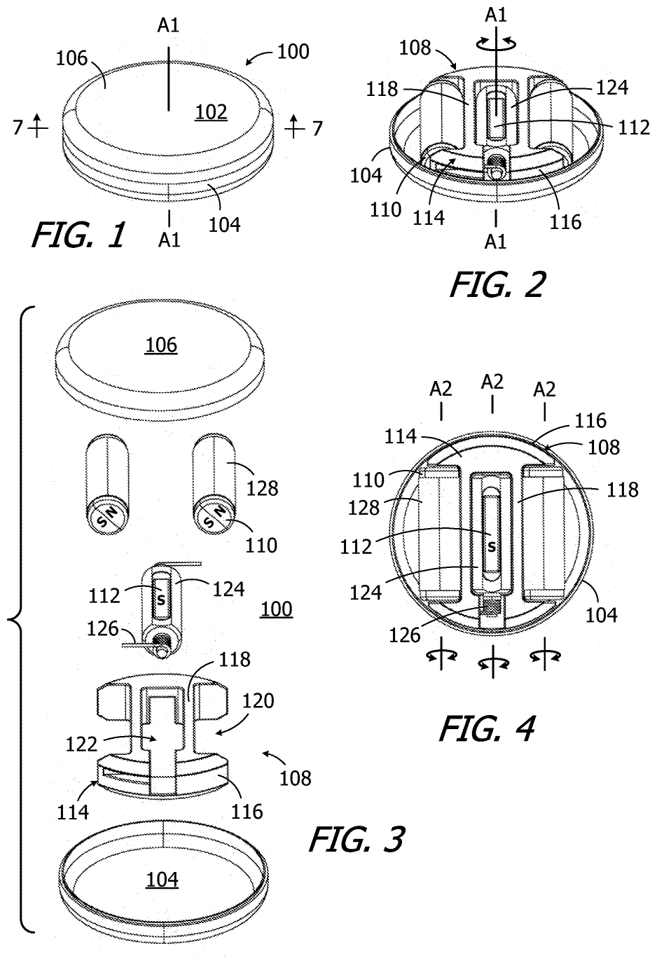

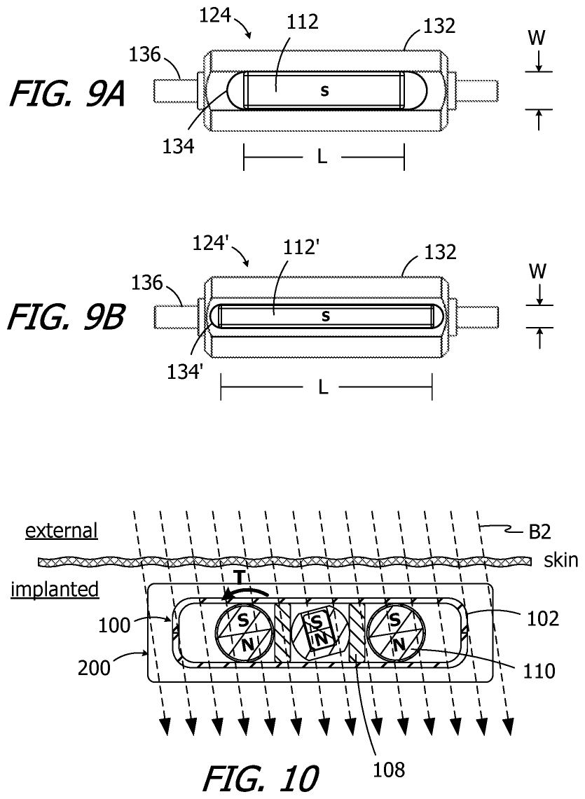

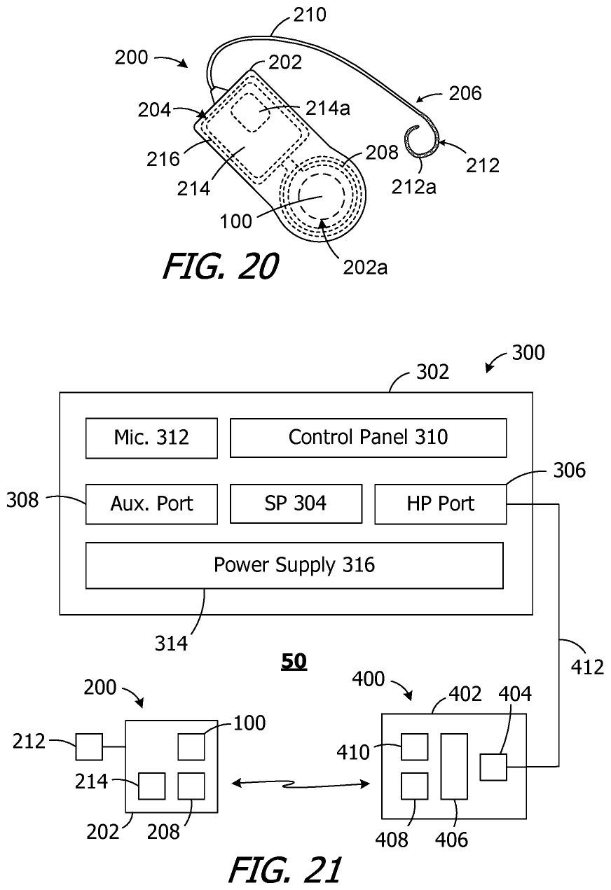

As illustrated for example in FIGS. 1-4, an exemplary magnet apparatus (or “magnet assembly”) 100 includes a case 102, with base 104 and a cover 106, a frame 108 that is rotatable relative to the case, and first and second elongate diametrically magnetized magnets 110 that are rotatable relative to the frame and that are not mechanically biased to respective N-S rotational orientations. In the illustrated embodiment, there are only two (i.e., two and no more than two) of the non-mechanically biased rotatable magnets 110. A third elongate magnet 112 is located between the first and second magnets 110, is rotatable relative to the frame, and is mechanically biased to a predetermined N-S rotational orientation. In particular, the elongate magnet 112 is mechanically biased to a N-S rotational orientation illustrated in FIGS. 1-7. As used herein, “mechanically biased” refers to the application of a biasing force by a one or more structural elements, at least one of which is in contact with the object that is being mechanically biased, and does not refer to magnetic force that may be acting on the object. Accordingly, an object that is not being mechanically biased to a predetermined N-S rotational orientation may nevertheless be held in a particular rotational orientation by magnetic force. The magnet assembly 100 may, in some instances, be employed in a system 50 (FIG. 9) that includes a cochlear implant 200 (described below with reference to FIG. 20) with a magnet assembly 100 and an external device such as a headpiece 400 (described below with reference to FIGS. 9 and 21).

The case 102 in the exemplary magnet apparatus 100 is disk-shaped and defines a central axis A1, which is also the central axis of the frame 108. The frame 108 is rotatable relative to the case 102 about the central axis A1 over 360°. The magnets 110 and 112 rotate with the frame 108 about the central axis A1. Each magnet 110 is also rotatable relative to the frame 108 about its own longitudinal axis A2 (also referred to as “axis A2”) over 360°. The magnet 112 is also rotatable relative to the frame 108 about its own longitudinal axis A2, albeit over less than over 360° due to the mechanical biasing structures (discussed below). In the exemplary implementation illustrated in FIGS. 1-8, the longitudinal axes A2 are parallel to one another and are perpendicular to the central axis A1. In other implementations, the magnets may be oriented such that the longitudinal axes thereof are at least substantially perpendicular to the central axis A1. As used herein, an axis that is “at least substantially perpendicular to the central axis” includes axes that are perpendicular to the central axis as well as axes that are slightly non-perpendicular to the central axis (i.e., axes that are offset from perpendicular by up to 5 degrees).

The exemplary case 102 is not limited to any particular configuration, size or shape. In the illustrated implementation, the case 102 is a two-part structure that includes the base 104 and the cover 106 which are secured to one another in such a manner that a hermetic seal is formed between the cover and the base. Suitable techniques for securing the cover 106 to the base 104 include, for example, seam welding with a laser welder. With respect to materials, the case 102 may be formed from biocompatible paramagnetic metals, such as titanium or titanium alloys, and/or biocompatible non-magnetic plastics such as polyether ether ketone (PEEK), low-density polyethylene (LDPE), high-density polyethylene (HDPE), ultra-high-molecular-weight polyethylene (UHMWPE), polytetrafluoroethylene (PTFE) and polyamide. In particular, exemplary metals include commercially pure titanium (e.g., Grade 2) and the titanium alloy Ti-6Al-4V (Grade 5), while exemplary metal thicknesses may range from 0.20 mm to 0.25 mm. With respect to size and shape, the case 102 may have an overall size and shape similar to that of conventional cochlear implant magnets so that the magnet apparatus 100 can be substituted for a conventional magnet in an otherwise conventional cochlear implant. The case 102 may also have an overall size and shape that is larger than that of conventional cochlear implant magnets in other embodiments. In some implementations, the diameter that may range from 9 mm to 17.4 mm and the thickness may range from 1.5 mm to 4.0 mm. The diameter of the case 102 in the illustrated embodiment is about 12.7 mm and the thickness is about 3.1 mm. As used herein in the context of the case 102, the word “about” means ±10%.

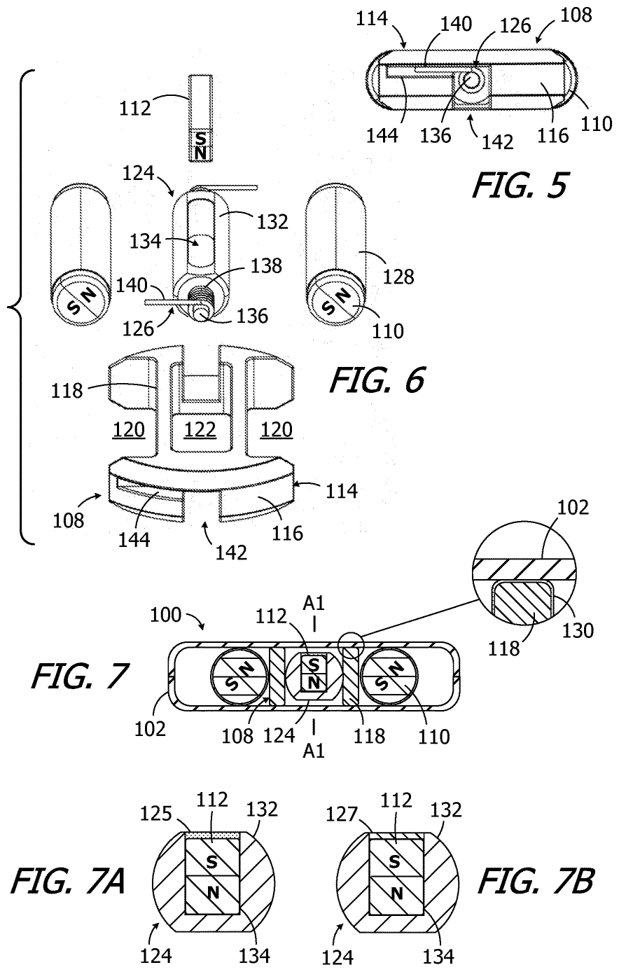

The exemplary frame 108 includes first and second base members 114, which have curved ends 116, and first and second bars 118 that extend from one base member to the other. The base members 114 and bars 118 together define receptacles 120 for the magnets 110 and a receptacle 122 for the magnet 112. In the illustrated implementation, the magnet 112 is supported within the frame receptacle 122 by a magnet holder 124 and resilient elements such as springs 126, as is discussed below with reference to FIGS. 5 and 6. Suitable materials for the frame 108, which may be formed by machining, metal injection molding or injection molding, include paramagnetic metals, polymers and plastics such as those discussed above in the context of the case 102. The magnet holder 124 may be formed from materials such as, for example, titanium, titanium alloys, MP35 alloy, Elgiloy® cobalt-chromium-nickel-molybdenum alloy, stainless steel and other non-magnetic metals. Referring more specifically to FIG. 4, there may be a relatively tight fit between the between the magnets 110 and the receptacles 120. For example, the length of the receptacles 120 may be about 0.05 mm to about 0.20 mm greater than the length of the magnets 110 in some implementations. As used herein in the context of the frame, the word “about” means ±10%.

The magnets 110 in the exemplary magnet apparatus 100 are elongate diametrically magnetized magnets, and there are only two magnets 110 within the case 102. The exemplary magnets 110 are circular in a cross-section that is perpendicular to the longitudinal axis A2 and, in some instances, may have rounded corners. Although not so limited, the magnet 112 in exemplary embodiment is rectangular in a cross-section that is perpendicular to the longitudinal axis A2. Suitable materials for the magnets 110 and 112 include, but are not limited to, neodymium-boron-iron and samarium-cobalt. In the illustrated embodiment, the magnets 110 and 112 are N52 or N55 neodymium-boron-iron magnets. The length of the magnets 110 may range from about 5.0 mm to about 8.3 mm and is about 8.3 mm in the illustrated embodiment, while the diameter of the magnets 110 may range from about 1.5 mm to about 2.4 mm and is about 2.4 mm in the illustrated embodiment. The magnet 112 is smaller than the magnets 110. The length of the magnet 112 may range from about 3.0 mm to about 7.0 mm and is about 4.5 mm in the illustrated embodiment, the height (in the N-S direction) may range from about 1.0 mm to about 1.8 mm and is about 1.6 mm in the illustrated embodiment, and the width (perpendicular to the N-S direction) may range from about 1.0 mm to about 1.5 mm and is about 1.0 mm in the illustrated embodiment. Put another way, the volume of the magnet 112 may range from about 15% to about 30% of the volume of one of the magnets 110, and is about 18% of the volume of one of the magnets 110 in the illustrated embodiment. As used herein in the context of the magnet size, the word “about” means ±10%. The frame 108 maintains the maintains the spacing between the magnets 110, as well as between the magnets 110 and the magnet 112.

The magnets 110 may be located within tubes 128 formed from low friction material. Suitable materials for the tubes 128 include polymers, such as silicone, PEEK and other plastics, PTFE, and PEEK-PTFE blends, and paramagnet metals. The magnets 110 may be secured to the tubes 128 such that the each tube rotates with the associated magnet about its axis A2, or the magnets may be free to rotate relative to the tubes. The magnet/tube combination is also more mechanically robust than a magnet alone. The magnets 110 may, in place of the tubes 128, be coated with the lubricious materials discussed below.

Friction may be further reduced by coating the inner surfaces of the case 102 and/or the surfaces of the frame 108 with a lubricious layer. The lubricious layer may be in the form of a specific finish of the surface that reduces friction, as compared to an unfinished surface, or may be a coating of a lubricious material such as diamond-like carbon (DLC), titanium nitride (TiN), PTFE, polyethylene glycol (PEG), Parylene, fluorinated ethylene propylene (FEP) and electroless nickel sold under the tradenames Nedox® and Nedox PF™. The DLC coating, for example, may be only 0.5 to 5 microns thick. In those instances where the base 104 and a cover 106 are formed by stamping, the finishing process may occur prior to stamping. Micro-balls, biocompatible oils and lubricating powders may also be added to the interior of the case to reduce friction. In the illustrated implementation, the surfaces of the frame 108 may be coated with a lubricious layer 130 (e.g., DLC), while the inner surfaces of the case 102 do not include a lubricious layer, as shown in FIG. 7. The lubricious layer 130 reduces friction between the case 102 and frame 108.

As noted above, the elongate magnet 112 is mechanically biased to the N-S rotational orientation illustrated in FIGS. 1-7, where the N-S direction of the magnet is the same as (or is parallel to) the direction of axis A1. The mechanical biasing may be accomplished with any suitable instrumentality. Referring to FIGS. 5-7, in the exemplary magnet apparatus 100, the magnet 112 is located within and is permanently secured to a magnet holder 124 which includes an elongate body 132 (that may be circular, oval, square or other shapes in a cross-section perpendicular to axis A2), a magnet receptacle 134 and axles 136 (one visible) that extend from each longitudinal end of the elongate body. The magnet 112 may be permanently secured to the magnet holder 124 through the use of epoxy resin or other suitable adhesive 125 (FIG. 7A) that is omitted from some figures so that the magnet 112 is visible. Alternatively, or in addition, the magnet 112 may be secured in place through the use of cover 127 (e.g., a titanium cover) that is welded to the magnet holder 124 in the manner illustrated in FIG. 7B. The cover 127 may extend over the entire length of the magnet 112, or over only a portion of the length. A tube, such as one of the above-described low friction tubes 128, may also be used as a cover. The magnet 112 may be embedded within the magnet holder in other instances. The springs 126 apply biasing force to magnet 112 by way of the magnet holder 124. The springs 126 may be coiled torsion springs, as shown, or any other suitable spring. Suitable spring materials include, but are not limited to, Nitinol and nickel-cobalt alloys. In at least some instances, the magnet 112 (and magnet holder 124) may rotate up to 90 to 150 degrees and generate a torque of 5 millinewton-meters (mNm) to 30 mNm.

Each spring 126 in the illustrated embodiment includes an end 138 that is permanently secured to elongate body 132 and/or the associated axle 136 (e.g., by welding) as well as an arm 140. The exemplary frame 108 is configured to support the magnet holder 124 and springs 126 in the orientation illustrated in FIGS. 1-7. In addition to the receptacle 122, the exemplary frame 108 includes a pair of recesses 142 for the axles 136 and the coiled portions of the springs 126 as well as a pair of receptacles 144 (one shown) for the spring arms 140. The respective configurations of the spring arms 140 and receptacles 144 result in the springs 126 being secured to the frame 108 and the prevention of rotation of the arm-side ends of the springs 126 relative to the frame.

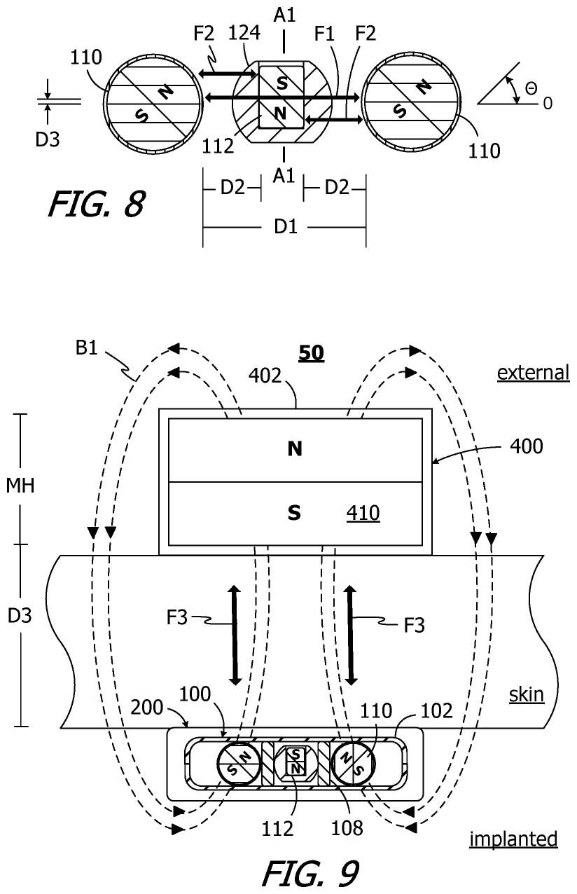

In the absence of a dominant magnetic field (such as an MRI magnetic field), the biasing force associated with the springs 126 maintains the magnet 112 in the illustrated orientation with N-S direction that is the same as the direction of axis A1 as shown in FIG. 1-7. Referring to FIG. 8, the distance D1 between the magnets 110 may range from about 3.8 mm to about 4.2 mm and is about 4.0 mm in the illustrated embodiment, while the distance D2 between the between the magnets 110 and the magnet 112 may range from about 1.2 mm to about 1.6 mm and is about 1.5 mm in the illustrated embodiment. As used herein in the context of the magnet spacing, the word “about” means ±10%. Absent the magnet 112, the magnets 110 would align with one another S-N-S-N horizontally in the illustrated orientation (i.e., parallel to the implant antenna plane and parallel to a plane perpendicular to axis A1) due to the magnetic force F1, despite the fact the magnetic force F1 is relatively weak as a result of the relatively large distance D1. The presence of magnet 112 and the associated magnetic forces F2 between the magnet 112 and the magnets 110, as well as the mechanical biasing force that maintains the magnet 112 in the illustrated rotational orientation with the N-S direction being the same as the axis A1 direction (i.e., perpendicular to the implant antenna plane and perpendicular to a plane perpendicular to axis A1), results in the magnets 110 each being rotationally offset from a plane perpendicular to axis A1 by an angle Θ that ranges from about 20° to about 45° in the absence of a dominant magnetic field. The magnet 112 also centralizes the magnetic field of the magnet apparatus 100. In other words, the magnets 110 rotate toward the magnet 112, and strongest portion the magnetic fields of the magnets 110 is focused toward the center of the headpiece magnet.

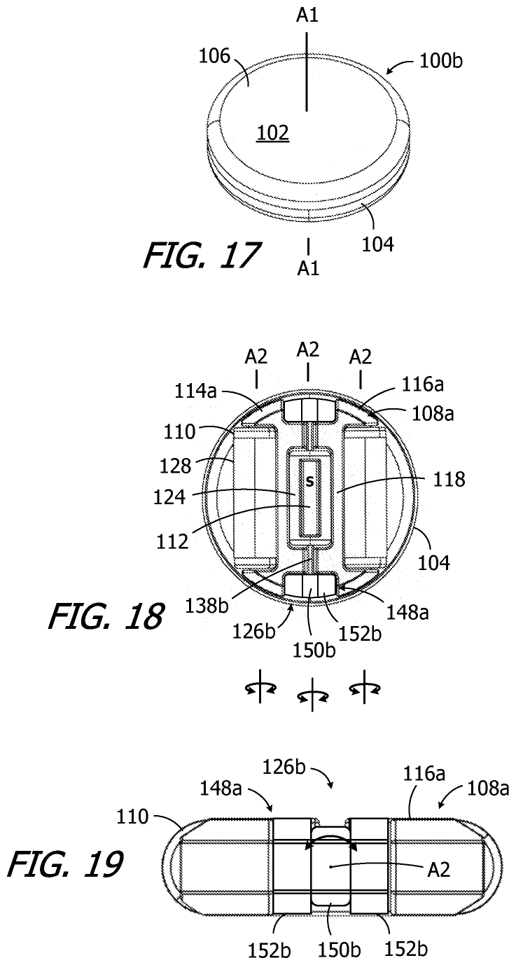

Turning to FIG. 9, the exemplary magnet apparatus (or “magnet assembly”) 100 may be part of an implanted cochlear implant 200 with a housing 202 (described in detail below with reference to FIG. 17) that is employed in conjunction with an external device such as a headpiece 400 (described in detail below with reference to FIG. 18) in a system 50. The cochlear implant 200 and headpiece 400 are shown on opposite sides of a relatively thick (i.e., 10 mm or more) skin flap. The exemplary headpiece 400 includes, among other things, a housing 402 and an axially magnetized disk-shaped positioning magnet (or “external magnet”) 410. The N-S direction of the external magnet 410 is at least substantially perpendicular (i.e., is perpendicular ±5%) to the implant recipient's skin. Although not so limited, the exemplary axially magnetized magnet 410 may have a height MH of about 7.6 mm and a diameter of about 11.45 mm. It should also be noted that the implant 200 and headpiece 400 are configured such that like poles of the headpiece magnet 410 and the mechanically biased implant magnet 112 face one another (e.g., S-S as shown) across the skin.

The respective configurations of the magnet assembly 100 and the headpiece 400 are such that when the implanted magnets 110 are exposed to the magnetic field B1 of the axially magnetized external magnet 410 across the relatively thick skin flap, the magnetic attraction force F3 between the external magnet 410 and an implanted magnet 110 is greater than the sum of the magnetic attraction force F1 between the two magnets 110 and the magnetic attraction force F2 between that magnet 110 and the magnet 112. The magnetic attraction force F3 may be, for example, at least 10% greater than the combined magnetic attraction forces F1+F2, or may be, for example, at least 20% greater than the combined magnetic attraction forces F1+F2. As a result, the magnets 110 advantageously rotate from the state illustrated in FIGS. 1-8 into alignment with the magnetic field B1 of the axially magnetized external magnet 410, as shown in FIG. 9. Put another way, the individual magnetic dipole moments of the elongate diametrically magnetized implant magnets 110 are oriented substantially in the direction of the magnetic field of the axially magnetized external magnet 410 during attractive transcutaneous magnetic interaction with the axially magnetized external magnet 410. The axially magnetized magnet 410 will also align with the center of the magnet apparatus 100, thereby aligning the headpiece antenna with the implant antenna. The magnets 110 will return to the state illustrated in FIGS. 1-8, due to the presence of the magnet 112, when the headpiece 400 and the associated magnetic field B1 is removed.

Another aspect of the exemplary magnet apparatus 100 is the impact resistance associated with the locations of the elongate diametrically magnetized magnets 110. When the magnet apparatus 100 is subjected to an impact force (e.g., when the user bumps his/her head), the central portion of the case 102 will deflect inwardly. Advantageously, the magnets 110 are offset from the central axis A1 of the case 102 by one-half of the distance D1 (FIG. 8), which reduces the likelihood of damage to the magnets as compared to a similar magnet apparatus where at least some of the magnets are located at or near the central axis A1. The magnet 112 is protected the magnet holder 124.

It should be noted here that although the diametrically magnetized magnets 110 are identical to one another, are parallel to one another, and are equidistant from the central axis A1 of the case 102 in the illustrated embodiment, the present magnet apparatus are no so limited. By way of example, but not limitation, the diametrically magnetized magnets 110 may have different lengths and/or may have different diameters and/or may be formed from materials having the same or different strength. Alternatively, or in addition, the diametrically magnetized magnets 110 may be non-parallel, and be different distances from the central axis A1 of the case 102. The configurations of the receptacles 114 would be adjusted to accommodate these differences.

The magnet 112, as well as the relationships between the magnet 112 and the other aspects of the exemplary magnet apparatus 100, are not limited to those described above. By way of example, but not limitation, the N-S center of the magnet 112 is offset from centers of the magnets 110 (i.e., is offset from the plane defined by the axes A2) by as distance D3 (FIG. 8). The distance D3 is about 0.2 mm in the illustrated embodiment and may be increased or decreased to adjust the manner in which the magnets 110 interact with the headpiece magnet 410 or other headpiece magnet. Alternatively, or in addition, the size, shape and/or volume of the magnet 112 may be varied. Referring to FIG. 9A, the magnet 112 in the illustrated implementation defines a length L and a width W, and exemplary lengths and widths are discussed above. The length may be increase, and the width may be decreased, in other implementations. To that end, and referring to FIG. 9B, the magnet 112′ is within and is permanently secured to a magnet holder 124′ which includes an elongate body 132 and a magnet receptacle 134′. The exemplary magnet 112′ has a greater length and a lesser width than the magnet 112. The magnet 112′ and magnet holder 124′ may, for example, by incorporated into the magnet apparatus 100 in place of the magnet 112 and magnet holder 124. The longer magnet 112′ reduces the interaction between the magnets 110, as compared to the shorter magnet 112.

In any case, when exposed to a dominant MRI magnetic field B2 (FIG. 10), the torque T on the magnets 110 and 112 will rotate the magnets about their axis A2 (FIG. 4), and overcome the mechanical biasing force associated with springs 126, thereby aligning the magnetic fields of the magnets 110 and 112 with the MRI magnetic field B2. The frame 108 will also rotate about axis A1 as necessary to align the magnetic fields of the magnets 110 and 112 with the MRI magnetic field B2. When the magnet apparatus 100 is removed from the MRI magnetic field B2, the magnet 112 will return to its mechanically biased orientation, and the magnetic attraction between the magnets 110 and 112 will cause the magnets 110 to rotate about axis A2 back to the orientation illustrated in FIGS. 1-8.

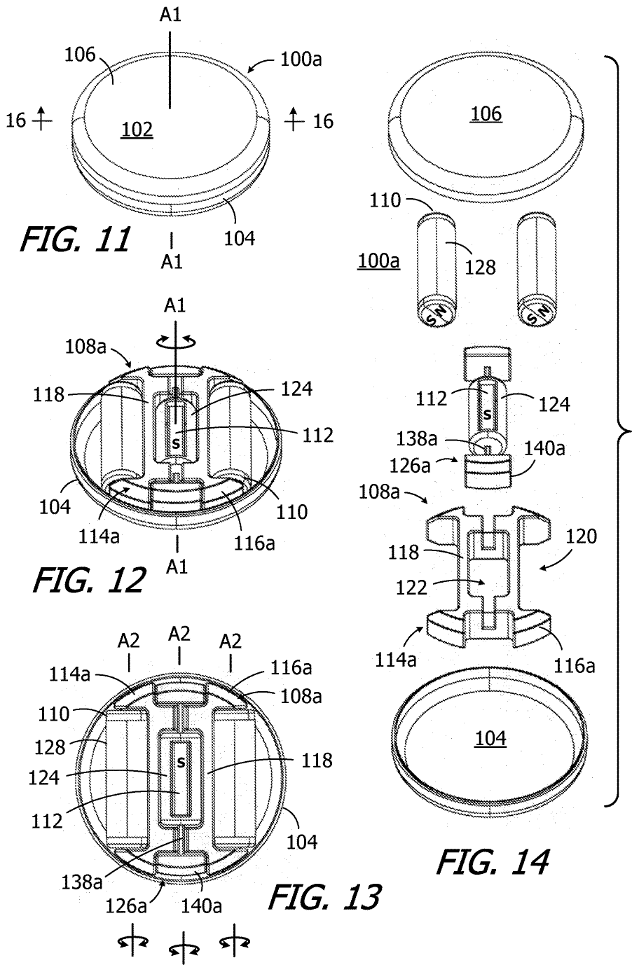

Another exemplary magnet apparatus (or “magnet assembly”) is generally represented by reference numeral 100a in FIGS. 11-16. Magnet apparatus 100a is substantially similar to magnet apparatus 100 and similar elements are represented by similar reference numerals. For example, the magnet apparatus 100a includes a case 102, with a base 104 and a cover 106, a rotatable frame 108a, two non-mechanically biased rotatable magnets 110 and a mechanically biased rotatable magnet 112. The frame 108a includes receptacles 120 for the magnets 110 and a receptacle 122 for the magnet 112, and the magnet 112 is supported within the frame receptacle 122 by a magnet holder 124.

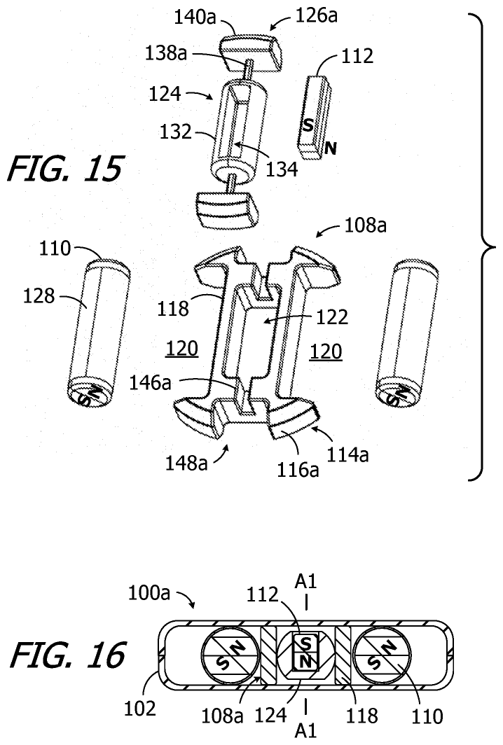

Here, however, the resilient elements that mechanically bias the magnet 112 to the N-S rotational orientation illustrated in FIGS. 11-16 are torsion rod assemblies 126a. The torsion rod assemblies 126a each include a torsion rod 138a and an anchor 140a. One end of each torsion rod 140a is connected to the magnet holder 124 and the other end is connected to one of the anchors 140a. Suitable materials for the torsion rod assemblies 126a, as well as the holder 124 and the torsion rod assemblies when they are a one-piece molded part, include but are not limited to rubber, polymers and plastics. The frame 108a is configured to receive the torsion rod assembly 126a. In particular, and referring more specifically to FIG. 15, the frame 108a includes a pair of channels 146a for the torsion rods 138a and a pair of anchor receptacles 148a for the anchors 140a. The respective configurations of the anchors 140a and receptacles 148a result in the torsion rod assemblies 126a being secured to the frame 108a and the prevention of rotation of the anchor-side ends of the torsion rods 138a relative to the frame. The respective configurations of the magnets 110 and 112 and the torsion rod assemblies 126a in magnet apparatus 100a are such that the magnets 110 and 112 will function in the manner described above (in the context of magnet apparatus 100) with respect to one another, with respect to the headpiece magnet 410, and with respect to a dominant MRI magnetic field. For example, the torsions rods 138a will maintain the magnet 112 in the orientation illustrated in FIGS. 11-16 in absence an MRI magnetic field, will twist and allow the magnet 112 to rotate into alignment with the MRI magnetic field, and will return the magnet 112 to the illustrated orientation when the MRI magnetic field is removed.

It should be noted that other resilient elements may be used in place of the torsion rods 138a. By way of example, but not limitation, the exemplary magnet apparatus 100b illustrated in FIGS. 17-19 is substantially similar to magnet apparatus 100a and similar elements are represented by similar reference numerals. Here, however, torsion assemblies 126b have taken the place of the torsion rod assemblies 126a. The torsion assemblies 126b each include a rigid rod 138b that rotates with magnet holder 124 (as opposed to a twistable torsion rod), an arm 150b that is mounted on the end of the rigid rod and is within the associated receptacle 148a, and a pair of resilient blocks 152b that are within the receptacle 148a on opposite sides of the arm. Suitable materials for the resilient blocks include, but are not limited to, compressible foam and dense rubber. The modulus of the resilient blocks 152b is sufficient to hold the arm 150b and, therefore, the rigid rod 138b, the magnet holder 124 and the magnet 112, in the illustrated orientation in the absence of an MRI magnetic field. Portions of the resilient blocks 152b will be compressed by the arms 150b when the presence of an MRI magnetic field causes rotation of the magnet 112 and, therefore, rotation of the arms 150b about axis A. The resilience of the blocks will return the magnet 112 to the illustrated orientation when the MRI magnetic field is removed.

One example of a cochlear implant (or “implantable cochlear stimulator”) including the present magnet apparatus 100 (or 100a or 100b) is the cochlear implant 200 illustrated in FIG. 20. The cochlear implant 200 includes a flexible housing 202 formed from a silicone elastomer or other suitable material, a processor assembly 204, a cochlear lead 206, and an antenna 208 that may be used to receive data and power by way of an external antenna that is associated with, for example, a sound processor unit. The cochlear lead 206 may include a flexible body 210, an electrode array 212 at one end of the flexible body, and a plurality of wires (not shown) that extend through the flexible body from the electrodes 212a (e.g., platinum electrodes) in the array 212 to the other end of the flexible body. The magnet apparatus 100 is located within a region encircled by the antenna 208 (e.g., within an internal pocket 202a defined by the housing 202) and insures that an external antenna (discussed below) will be properly positioned relative to the antenna 208. The exemplary processor assembly 204, which is connected to the electrode array 212 and antenna 208, includes a printed circuit board 214 with a stimulation processor 214a that is located within a hermetically sealed case 216. The stimulation processor 214a converts the stimulation data into stimulation signals that stimulate the electrodes 212a of the electrode array 212.

Turning to FIG. 21, the exemplary cochlear implant system 50 includes the cochlear implant 200, a sound processor, such as the illustrated body worn sound processor 300 or a behind-the-ear sound processor, and a headpiece 400.

The exemplary body worn sound processor 300 in the exemplary ICS system 50 includes a housing 302 in which and/or on which various components are supported. Such components may include, but are not limited to, sound processor circuitry 304, a headpiece port 306, an auxiliary device port 308 for an auxiliary device such as a mobile phone or a music player, a control panel 310, one or more microphones 312, and a power supply receptacle 314 for a removable battery or other removable power supply 316 (e.g., rechargeable and disposable batteries or other electrochemical cells). The sound processor circuitry 304 converts electrical signals from the microphone 312 into stimulation data. The exemplary headpiece 400 includes a housing 402 and various components, e.g., a RF connector 404, a microphone 406, an antenna (or other transmitter) 408 and an axially magnetized disk-shaped positioning magnet 410, that are carried by the housing. The headpiece 400 may be connected to the sound processor headpiece port 306 by a cable 412. The external positioning magnet 410 is attracted to the magnet apparatus 100 of the cochlear stimulator 200 (see FIG. 6), thereby aligning the antenna 408 with the antenna 208. The stimulation data and, in many instances power, is supplied to the headpiece 400. The headpiece 400 transcutaneously transmits the stimulation data, and in many instances power, to the cochlear implant 200 by way of a wireless link between the antennae. The stimulation processor 214a converts the stimulation data into stimulation signals that stimulate the electrodes 212a of the electrode array 212.

In at least some implementations, the cable 412 will be configured for forward telemetry and power signals at 49 MHz and back telemetry signals at 10.7 MHz. It should be noted that, in other implementations, communication between a sound processor and a headpiece and/or auxiliary device may be accomplished through wireless communication techniques. Additionally, given the presence of the microphone(s) 312 on the sound processor 300, the microphone 406 may be also be omitted in some instances.

The functionality of the sound processor 300 and headpiece 400 may also be combined into a single head wearable sound processor that includes all of the external components (e.g., the battery, microphone, sound processor, antenna coil and magnet). Examples of head wearable sound processors are illustrated and described in U.S. Pat. Nos. 8,811,643 and 8,983,102, which are incorporated herein by reference in their entirety. Headpieces and head wearable sound processors are collectively referred to herein as “head wearable external components.”

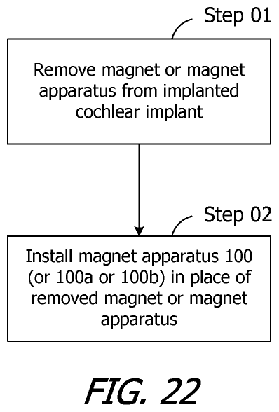

The present inventions are applicable to systems that include cochlear implants which have already been implanted into the recipient. For example, a similarly sized magnet, or a magnet apparatus with a similarly sized case, may be removed in situ from an implanted cochlear implant (Step 01) in the exemplary method illustrated in FIG. 22. In some instances, the magnet or magnet apparatus may be removed from a pocket in the cochlear implant housing. The exemplary magnet apparatus 100 (or 100a or 100b) described herein may be installed in place of the removed magnet or magnet apparatus (Step 02). In some instances, the magnet apparatus 100 (or 100a or 100b) may be inserted into the same pocket in the cochlear implant housing from which magnet or magnet apparatus was removed. Suitable removal and installation tools and techniques are illustrated and described in U.S. Pat. No. 10,124,167, which is incorporated herein by reference in its entirety. The headpiece magnet in the associated system may, if necessary, be removed from the headpiece or other head wearable external component and replaced with an axially magnetized magnet.

Although the inventions disclosed herein have been described in terms of the preferred embodiments above, numerous modifications and/or additions to the above-described preferred embodiments would be readily apparent to one skilled in the art. The inventions include any combination of the elements from the various species and embodiments disclosed in the specification that are not already described. It is intended that the scope of the present inventions extend to all such modifications and/or additions and that the scope of the present inventions is limited solely by the claims set forth below.

1. A magnet assembly, comprising:
a case defining a central axis;
a magnet frame within the case and rotatable about the central axis of the case;
first and second elongate magnets, located within in the frame, that are diametrically magnetized, that each define a longitudinal axis and a N-S direction, that are rotatable about the longitudinal axis relative to the frame, that are separated from one another by a fixed non-zero distance that is perpendicular to at least one of the longitudinal axes, and that are not mechanically biased to respective N-S rotational orientations; and
a third elongate magnet, located between the first and second elongate magnets, that defines a longitudinal axis and a N-S direction that is perpendicular to the longitudinal axis, and that is mechanically biased to a predetermined N-S rotational orientation.

2. A magnet assembly as claimed in claim 1, wherein
the predetermined N-S rotational orientation of the third elongate magnet is parallel to the central axis.

3. A magnet assembly as claimed in claim 1, wherein
the third elongate magnet defines a length in the direction of the longitudinal axis, a width that is less than the length and a thickness that is less than the length.

4. A magnet assembly as claimed in claim 1, wherein
the third elongate magnet is carried by a rotatable magnet holder.

5. A magnet assembly as claimed in claim 1, wherein
the third elongate magnet is mechanically biased by at least one resilient element.

6. A magnet assembly as claimed in claim 5, wherein
the resilient element comprises a spring.

7. A magnet assembly as claimed in claim 5, wherein
the resilient element comprises a torsion rod or a resilient block.

8. A magnet assembly as claimed claim 5, wherein
the magnet holder includes an elongate body and a magnet receptacle in the elongate body; and
the resilient element includes a first portion that is secured to the frame and a second portion that is secured to the rotatable magnet holder.

9. A magnet assembly as claimed in claim 1, wherein
the third magnet is smaller than the first magnet and is smaller than the second magnet.

10. A magnet assembly as claimed in claim 1, wherein
the first, second and third magnets define respective volumes; and
the volume of third magnet is about 15% to about 30% of the volume of the first magnet and is about 15% to about 30% of the volume of the second magnet.

11. A magnet assembly as claimed claim 2, wherein
absent a dominant magnetic field, the respective N-S directions of the first and second magnets are not perpendicular to the N-S direction of the third magnet.

12. A magnet assembly as claimed in claim 1, wherein
absent a dominant magnetic field, the respective N-S directions of the first and second magnets are rotationally offset from a plane perpendicular to the central axis.

13. A magnet assembly as claimed in claim 1, wherein
absent a dominant magnetic field, the respective N-S directions of the first and second magnets are rotationally offset from a plane perpendicular to the central axis by about 20° to about 45°.

14. A magnet assembly as claimed in claim 1, wherein
the first and second elongate magnets are the only magnets that are not mechanically biased to a N-S rotational orientation.

15. A system, comprising:
a cochlear implant having
a cochlear lead including a plurality of electrodes,
an implant antenna,
an implant processor operably connected to the implant antenna and to the cochlear lead, and
a magnet assembly as claimed in claim 1 adjacent to the implant antenna; and
a head wearable external component including an axially magnetized external magnet and an external antenna adjacent to the axially magnetized external magnet

16. A system as claimed in claim 15, wherein
the third elongate magnet and the external magnet define respective N-poles and S-poles;
the predetermined N-S rotational orientation of the third elongate magnet and the orientation of the external magnet within the external component are such that the like poles of the third elongate magnet and the external magnet face one another and are the poles that are closest to one another when the external magnet is attracted to the magnet assembly.

17. A method, comprising
removing a magnet or a magnet apparatus from an implanted cochlear implant; and
installing a magnet apparatus as described in claim 1 in place of the removed magnet or magnet apparatus.

18. A method as claimed in claim 17, further comprising:
removing a magnet from a head wearable external component; and
replacing the magnet removed from the head wearable external component with an axially magnetized magnet.
